(19) United States
(12) Patent Application Publication
(10) Pub. No.: US 2023/0013415 A1
(43) Pub. Date: Jan. 19, 2023

(54) LIGHTING DEVICE

(71) Applicant: Frido Technologies Limited, London (GB)

(72) Inventors: James FELLOWES, London (GB); Harry TRIGGS, London (GB); George SKINITIS, London (GB); Andrew WEBB, London (GB); Michael URDANG, London (GB)

(21) Appl. No.: 17/864,203

(22) Filed: Jul. 13, 2022

(30) Foreign Application Priority Data
Jul. 16, 2021 (GB) 2110245.4

(51) Int. Cl. A61L 2/10

(52) U.S. Cl. CPC A61L 2/10; A61L 2202/11; A61L 2202/14

(57) ABSTRACT

Aspects described herein include methods, apparatuses, and non-transitory computer-readable storage mediums for operating a germicidal lighting device. A target environment is monitored for one or more people. In response to the monitoring indicating the presence of one or more people, the lighting device is caused to operate in an occupied target environment operating mode including causing the lighting device to emit far-UVC light at a given intensity into the target environment for a first period of time; and, in response to expiry of the first period of time, causing the lighting device to emit far-UVC light at a different intensity to the given intensity into the target environment for a second period of time.

### CROSS-REFERENCE TO RELATED APPLICATIONS

This application claims priority under 35 U.S.C. § 119(a) to UK Patent Application No. 2110245.4, filed on 16 Jul. 2021, the entire content of which is hereby incorporated by reference.

### INTRODUCTION

The present disclosure concerns germicidal lighting devices. More particularly, but not exclusively, the present disclosure concerns germicidal lighting devices configured to emit far-UVC light and methods of operating such lighting devices.

Ultraviolet-C (UVC) light, due to its germicidal properties, has long been used to disinfect objects, surfaces and air. In particular, the use of far-UVC light (UVC light having a wavelength of between 207 nm and 222 nm) in such a manner is known. It will be appreciated by the skilled person that the term germicidal refers to the ability of UVC light (including far-UVC light) to inactivate microorganisms such as bacteria, viruses, and protozoa. In particular, UVC light has previously been used to sterilize hospital spaces. However, the use of UVC light in this way has fallen out of favor, following increased use of antibiotics. Overexposure to UVC light is closely linked to adverse effects on human health, including skin cancer and cataract induction. Therefore, today, UVC light is typically only used in unoccupied spaces.

Furthermore, far-UVC light sources (for example, excimer lamps) typically have short lifespans (for example, of up to approximately 5000 hours of use). Therefore, extensive and/or widespread use of far-UVC germicidal lighting devices can lead to high maintenance and cost requirements.

The present disclosure seeks to mitigate the above-mentioned problems. Alternatively or additionally, the present disclosure seeks to provide an improved germicidal lighting device and method of operating such a lighting device.

### SUMMARY

According to a first aspect of the present disclosure, there is provided a method of operating a germicidal lighting device, the method comprising: monitoring a target environment for one or more people; and in response to the monitoring indicating the presence of one or more people, causing the lighting device to operate in an occupied target environment operating mode comprising: causing the lighting device to emit far-UVC light at a given intensity into the target environment for a first period of time; and in response to expiry of the first period of time, causing the lighting device to emit far-UVC light at a different intensity to the given intensity into the target environment for a second period of time.

According to a second aspect of the present disclosure, there is provided a germicidal lighting device comprising: a UVC light source configured to emit far-UVC light; an occupancy sensor configured to monitor a target environment for one or more people; and a controller configured to, in response to the monitoring indicating the presence of one or more people, cause the lighting device to operate in an occupied target environment operating mode comprising: causing the lighting device to emit far-UVC light at a given intensity into the target environment for a first period of time; and in response to expiry of the first period of time, causing the lighting device to emit far-UVC light at a different intensity to the given intensity into the target environment for a second period of time.

According to a third aspect of the present disclosure, there is provided a non-transitory computer-readable storage medium comprising computer-executable instructions which, when executed by a processor, cause a computerized device to perform a method of operating a germicidal lighting device, the method comprising: monitoring a target environment for one or more people; and in response to the monitoring indicating the presence of one or more people, causing the lighting device to operate in an occupied target environment operating mode comprising: causing the lighting device to emit far-UVC light at a given intensity into the target environment for a first period of time; and in response to expiry of the first period of time, causing the lighting device to emit far-UVC light at a different intensity to the given intensity into the target environment for a second period of time.

It will of course be appreciated that features described in relation to one aspect of the present disclosure may be incorporated into other aspects of the present disclosure. For example, the method of the present disclosure may incorporate any of the features described with reference to the apparatus of the present disclosure and vice versa.

### DESCRIPTION OF THE DRAWINGS

Embodiments of the present disclosure will now be described by way of example only with reference to the accompanying schematic drawings of which:

FIG. 1 shows a functional block diagram of a lighting device according to embodiments of the present disclosure;

FIGS. 2 and 3 show schematic views of lighting device installations according to embodiments of the present disclosure; and

FIGS. 4 to 6 show flow charts illustrating methods of operating a lighting device according to embodiments of the present disclosure.

### DETAILED DESCRIPTION

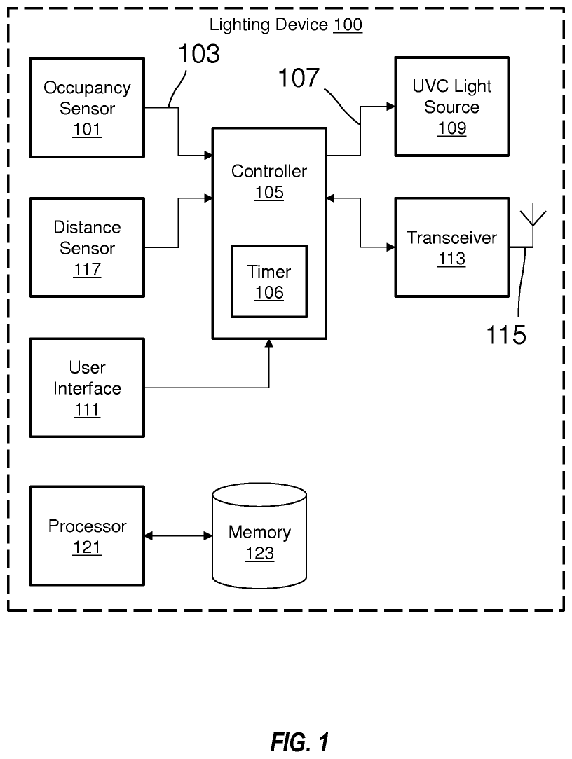

FIG. 1 shows a functional block diagram of a germicidal lighting device 100 according to embodiments of the present disclosure.

Lighting device 100 comprises an occupancy sensor 101. Occupancy sensor 101 is configured to monitor a target environment for one or more people. In embodiments, occupancy sensor 101 is configured to, in response to the monitoring indicating that one or more people are present in the target environment, generate a signal 103 indicating that the monitoring has detected one or more people within the target environment. When one or more people are present within the target environment, the target environment can be said to be occupied. When no people are present within the target environment, the target environment can be said to be unoccupied.

In embodiments, occupancy sensor 101 comprises one or more of: a passive infra-red (PIR) sensor, an ultrasonic sensor, and a sonar sensor, a light detection and ranging (LiDAR) sensor, and a radar sensor. In embodiments, occupancy sensor 101 comprises a radio frequency (RF) detector. In embodiments, such a detector may be configured to detect radio frequency radiation (for example, in the form of wireless signals) emitted by a source of radio frequency radiation. Such sources include wearable tags (for example, radio-frequency identification (RFID) tags), mobile phones, tablet computers, and other computing devices. It will be appreciated be the skilled person that other sensor types may, alternatively or additionally, be used to provide the functionality of occupancy sensor 101.

In embodiments, occupancy sensor 101 forms part of lighting device 100. In alternative embodiments, occupancy sensor 101 is separate from the rest of lighting device 100 (for example, being in a separate unit or installation). In such embodiments, it may be that occupancy sensor 101 is configured to communicate with lighting device 100 (for example, via a wired and/or wireless communications network) in order to convey signal 103 to lighting device 100.

In embodiments, the target environment comprises the space within which occupancy sensor 101 is capable of detecting a person. For example, in embodiments, lighting device 100 may be installed in an interior ceiling within a building, such that occupancy sensor 101 faces substantially downwards. In such embodiments, it may be that the target environment may comprise the space substantially beneath lighting device 100. It will therefore be appreciated by the skilled person that the particular range and extent of the target environment will be determined at least in part by the capabilities of occupancy sensor 101 (for example, its field of view and detection range). In embodiments, the target environment comprises one or more of an interior of a boat, hospital, school, or other public building. In embodiments, the target environment comprises one or more of an elevator, reception area, or other interior public or private space or room.

Lighting device 100 further comprises a controller 105. In embodiments, controller 105 is configured to receive signal 103. In such embodiments, it may be that controller 105 is configured to, in response to signal 103 indicating the presence of one or more people in the target environment, generate a control signal 107 for use in controlling a UVC light source 109.

UVC light source 109 is configured to emit far-UVC light into the target environment. In embodiments, UVC light source 109 is configured to receive control signal 107 and to emit far-UVC light on the basis of control signal 107. Thus, controller 105 is configured to control UVC light source 109. Far-UVC light will be understood by the skilled person to comprise wavelengths of approximately 207-222 nm. In embodiments, UVC light source 109 is configured to emit UVC light other than far-UVC light. In alternative embodiments of the present disclosure, UVC light source 109 is configured to emit UVC light. In such alternative embodiments, it may be that UVC light source 109 is configured not to emit far-UVC light. In embodiments, UVC light source 109 is configured to emit light having a wavelength of between 207 nm and 225 nm. In embodiments, UVC light source 109 is configured to emit light having a wavelength of 207 nm and 222 nm. In embodiments, UVC light source 109 is configured to emit light having a wavelength of 222 nm (for example, light predominantly (for example, substantially only) having a wavelength of 222 nm). In embodiments, UVC light source 109 comprises an excimer lamp. In embodiments, UVC light source 109 comprises a plurality of UVC light sources. In such embodiments, it may be that UVC light source 109 is configured to control an intensity of far-UVC light emission (for example, by activating/inactivating one or more of the plurality of UVC light sources). In embodiments, UVC light source 109 is configured to control an intensity of far-UVC light emission by dimming the one or more UVC light sources (for example, by varying a duty cycle of light emission by UVC light source 109).

In embodiments, control signal 107 comprises a binary logic signal capable of having only two states (for example, a first state corresponding to activating UVC light source 109 and a second state corresponding to inactivating UVC light source 109). Alternatively, control signal 107 comprises control data configured to cause UVC light source 109 to change one or more parameters of far-UVC light emission by UVC light source 109. For example, in embodiments, control signal 107 comprises data configured to cause UVC light source 109 to alter one or more of: an intensity, a duration, and a direction of far-UVC light emission by UVC light source 109.

In embodiments, controller 105 is configured to control UVC light source 109 according to a number of different operating modes. Thus, lighting device 100 can be said to operate in multiple different operating modes.

Controller 105 is configured to, in response to the monitoring indicating the presence of one or more people, cause control lighting device 100 to operate in an occupied target environment operating mode. Operating in the occupied target environment operating mode comprises causing the lighting device to emit far-UVC light at a given (e.g. default) intensity into the target environment for a first period of time. Operating in the occupied target environment operating mode further comprises, in response to expiry of the first period of time, causing the lighting device to emit far-UVC light at a different (for example, reduced) intensity to the given intensity (for example, by ceasing to emit far-UVC light) into the target environment for a second period of time. Light emission by UVC light source 109 during the first period of time can be said to be at a default intensity. When UVC light source 109 is controlled to emit far-UVC light it can be said to be “active” and when UVC light source 109 is controlled to cease to emit far-UVC light it can be said to be “inactive”. In embodiments, controller 105 comprises a timer 106. In such embodiments, it may be that controller 105 is configured to operate timer 106 to monitor one or both of the first and second periods of time.

Thus, when lighting device 100 detects that one or more people are present within the target environment (and would therefore be exposed to far-UVC light from UVC light source 109), controller 105 operates to control (for example, reduce) the amount of time for which UVC light source 109 is emitting light at the default intensity (compared to if UVC light source 109 was active for the entire duration of time), and thereby reduces the far-UVC light exposure of the person. By controlling the ratio of the time UVC light source 109 is emitting far-UVC light at the default intensity to that at the different intensity (which can be referred to as a duty-cycle of UVC light source 109), it is possible to control the UVC exposure of people within the target environment (for example, to be within safe limits). Embodiments in which UVC lights source 109 is configured to cause UVC light source 109 to emit far-UVC light at a given (e.g. default) intensity into the target environment for a first period of time and, in response to expiry of the first period of time, cause UVC light source 109 to cease to emit far-UVC light into the target environment for a second period of time, can reduce the amount of time for which UVC light source 109 is active. This preserves and extends the operating life of UVC light source 109. The same also applies to embodiments in which the reduction in intensity of far-UVC light emission by light source 100 is achieved by activating or inactivating one or more of a plurality of UVC light sources.

In embodiments, at least one (for example, both) of the first period of time and the second period of time is dependent on a dimension of the target environment. It will be appreciated by the skilled person that the dosage of far-UVC light to which a person in the target environment is exposed will depend on a distance of the person from UVC light source 109. The dimensions of the target environment may affect the likely distance between UVC light source 109 and people within the target environment. For example, in embodiments in which lighting device 100 is installed within a ceiling, the height of the ceiling will affect the distance between UVC light source 109 and people within the target environment, and thereby also the dosage of far-UVC light accrued by those people from lighting device 100.

In embodiments, at least one (for example, both) of the first period of time and the second period of time is dependent on an intensity of far-UVC light emission by the lighting device. It will be appreciated by the skilled person that the dosage of far-UVC light to which a person in the target environment is exposed over a given time period will depend on an intensity of far-UVC light emission by UVC light source 109.

In embodiments, at least one (for example, both) of the first period of time and the second period of time is dependent on a predetermined maximum far-UVC light dosage rate. In such embodiments, it may be that the first period of time and/or the second period of time are selected in order to ensure that a person within the target environment is not exposed to far-UVC light in excess of the maximum far-UVC light dosage rate. In such embodiments, it may be that the predetermined maximum far-UVC light dosage rate is as determined by a safety regulation. In embodiments, the predetermined maximum far-UVC light dosage rate is 3mj/cm2/hr.

In embodiments, the first period of time and the second period of time are each longer than 0.01 seconds, for example longer than 0.1 seconds or longer than 1 second. In embodiments, the ratio of the first period of time to the second period of time is no greater than 1:1, for example no greater than 1:10, no greater than 1:25, or no greater than 1:50.

In embodiments, at least one (for example, both) of the first period of time and the second period of time are fixed (for example, having been set during installation or manufacture). Thus, in embodiments, one or both of the first period of time and the second period of time are predetermined. In embodiments, at least one (for example, both) of the first period of time and the second period of time are user-configurable. In embodiments, lighting device 100 is configured to receive user input indicative of the desired values for the first period of time and/or the second period of time. Thus, a user can adjust one or both of the first period of time and the second period of time (for example, in order to adapt to the geometry of the target environment).

In embodiments, the user input comprises an indication of a desired duty cycle of UVC light source 109. In embodiments, the user input comprises an indication of a desired length of one of the first period of time and the second period of time.

In embodiments, lighting device 100 comprises a user interface 111. In embodiments, user interface 111 is configured to enable a user to provide the user input. In embodiments, user interface 111 is configured to receive the user input indicative of the desired values for the first period of time and/or the second period of time. In embodiments, user interface 111 comprises one or more of a switch, a dial, a potentiometer, and a variable resistor. In embodiments, user input is provided via a graphical user interface (GUI). In embodiments, it may be that user interface 111 comprises a display (for example, on which the GUI is displayed). In other embodiments, the GUI is displayed on a separate computing device (for example, a laptop or a smartphone). It will be appreciated by the skilled person that other means for providing manual user input may also be used in other embodiments. In embodiments, the user input comprises an indication of a distance from lighting device 100 to a surface (for example, a floor) directly beneath lighting device 100.

In embodiments, lighting device 100 comprises a transceiver 113. In embodiments, transceiver 113 is configured to enable lighting device 100 to communicate over a communication link. In embodiments, the communication link is at least partially a wired communication link. In embodiments, the communication link is at least partially wireless. In such embodiments, it may be that lighting device 100 also comprises an antenna 115 for use in transmitting and/or receiving wireless transmitted signals. In embodiments, lighting device 100 receives the user input via a signal transmitted over a communication link (for example, received via transmitter 113 and/or antenna 115). In such embodiments, it may be that the user input is provided by the user to a separate computing device (for example, a laptop computer or a smartphone).

In embodiments, lighting device 100 is configured to communicate with one or more further instances of the lighting device (for example, via transceiver 113). In embodiments, lighting device 100 is configured to communicate to one or more such further lighting devices state information on a current state of lighting device 100. In embodiments, lighting device 100 is configured to control one or more further lighting devices (for example, to synchronize their operation to that of lighting device 100). Synchronizing the operation of a plurality of lighting devices can help to prevent a person being overexposed to far-UVC light as a result of having moved between the target environments of multiple nearby lighting devices. In such embodiments, it may be that lighting device 100 is a primary (or ‘controller’ or ‘master’) lighting device and the lighting devices with which lighting device 100 is in communication are secondary (or ‘agent’ or ‘slave’) lighting devices. In such embodiments, it may be that the agent lighting devices are configured to operate on the basis of commands and/or state data received from lighting device 100 (i.e. from the controller lighting device). Thus, in embodiments, lighting device 100 is configured to receive, from one or more further lighting devices, state information on current states of those lighting devices. In embodiments, lighting device 100 is configured to operate on the basis of the received state information. In embodiments, lighting device 100 is configured to receive control data from one or more further devices (for example, further germicidal lighting devices). In embodiments, lighting device 100 is configured to operate on the basis of the received control data. In embodiments, the one or more further devices comprise a control device (for example, configured to control and/or coordinate one or more germicidal lighting devices). In embodiments, the control device is not a germicidal lighting device. In embodiments, lighting device 100 is configured to be controlled by a further instance of the lighting device. In embodiments, lighting device 100 is configured to coordinate its operation with the one or more further lighting devices. In such embodiments, it may be that the coordination is performed via a peer-to-peer networking model (for example, in which no one lighting device is configured directly to control the other lighting devices).

In embodiments, controller 105 is configured to receive an indication of one or more dimensions of the target environment. In such embodiments, it may be that controller 105 is configured to determine, on the basis of the indicated one or more dimensions, the first period of time and/or the second period of time. In embodiments, determining the first period of time and/or the second period of time comprises retrieving from a lookup table one or more time periods associated with the indicated one or more dimensions.

In embodiments, controller 105 is configured to determine (for example, by use of occupancy sensor 101 or distance sensor 117) one or more distances from lighting device 100 to the one or more people in the target environment. In such embodiments, it may be that controller 105 is configured to determine, on the basis of the detected one or more distances, the first period of time and/or the second period of time. In embodiments, determining the first period of time and/or the second period of time comprises retrieving from a lookup table one or more time periods associated with the detected one or more distances. In embodiments in which occupancy sensor 101 comprises a radio frequency detector, lighting device 100 may be configured to use a detected distance to a source of radio frequency radiation as an approximation of the distance to the one or more people.

In embodiments, the indication of one or more dimensions of the target environment is received by user input (for example, via user interface 111 or transceiver 113). In alternative embodiments, lighting device 100 comprises a distance sensor 117. In such embodiments, it may be that distance sensor 117 is configured to determine the one or more dimensions. Thus, in embodiments, lighting device 100 is configured to operate one or more sensors to determine the one or more dimensions. In embodiments, distance sensor 117 comprises one or more of: a LiDAR sensor, a PIR sensor, an ultrasonic sensor, and a sonar. In such embodiments, lighting device 100 can autonomously determine and operate according to first and second periods of time which are suitable for the target environment in which it is deployed (for example, in order to avoid over-exposing people in the target environment to far-UVC light).

In embodiments, controller 105 is configured to, in response to the monitoring continuing to indicate the presence of one or more people, cause the lighting device to continue to operate in the occupied target environment operating mode. In embodiments, continuing to operate in the occupied target environment operating mode comprises repeating the causing the lighting device to emit far-UVC light at the given (e.g. default) intensity for the first period of time and the causing the lighting device to emit far-UVC light at the different intensity to the given intensity (for example, by ceasing to emit far-UVC light) for the second period of time.

Thus, in embodiments, lighting device 100 is configured to repeatedly, whilst the monitoring indicates that the target environment is occupied, emit far-UVC light at the given (e.g. default) intensity for the first period of time and emit far-UVC light at a different (for example, reduced) intensity to the given intensity (for example, by ceasing to emit far-UVC light) for the second period of time. Emitting far-UVC light for the first period of time followed by ceasing to emit far-UVC light for the second period of time results in UVC light source 109 emitting far-UVC light at a reduced average power. Thus, in embodiments, operating lighting device 100 in the occupied target environment operating mode comprises controlling UVC light source 109 to emit far-UVC light at a first average power. This results in a reduction in the exposure of people present in the target environment to far-UVC light. Thus, in embodiments, operating lighting device 100 in the occupied target environment operating mode comprises controlling UVC light source 109 such that the one or more people accrue far-UVC dosage at a predetermined dosage rate, for example a predetermined maximum dosage rate which is deemed safe.

In embodiments, controller 105 is configured to, in response to the monitoring indicating that the one or more people have vacated the target environment, cause lighting device 100 to operate in an unoccupied target environment operating mode. One or more people having vacated the target environment will be understood by the skilled person to mean the monitoring first indicating the presence of one or more people in the target environment (i.e. that the target environment is occupied) before subsequently indicating that no people are present in the target environment (i.e. that the target environment is subsequently unoccupied).

In embodiments, operating in the unoccupied target environment operating mode comprises controlling UVC light source 109 to emit far-UVC light differently to when operating in the occupied target environment operating mode. In such embodiments, it may be that one or more of a duty cycle, intensity, duration, and direction of far-UVC light emission in the unoccupied target environment operating mode differs from that of the occupied target environment operating mode. Thus, lighting device 100 can, when it is determined that no people are present in the target environment, emit far-UVC light differently to when people are present in the target environment. For example, in embodiments, lighting device 100 is configured to control UVC light source 109 to emit far-UVC light at an intensity or for a duration which would not be safe if a person was present in the target environment (for example, to provide increased sterilisation when the target environment is vacant).

In embodiments, an intensity (for example, an average intensity) of far-UVC light emission by UVC light source 109 in the occupied target environment operating mode is lower than an intensity of far-UVC light emission by UVC light source 109 in the unoccupied target environment operating mode. It will be appreciated by the skilled person that a change in the average intensity of far-UVC light emission can be achieved by a change in one or both of the instantaneous intensity and the duty cycle of far-UVC light emission by UVC light source 109. In embodiments, operating in the unoccupied target environment mode comprises causing the UVC light source 109 to emit far-UVC light at a maximum intensity (for example, for a further period of time).

In embodiments, operating lighting device 100 in the unoccupied target environment operating mode comprises causing UVC light source 109 to emit far-UVC light at a further given intensity into the target environment for a third period of time. In such embodiments, it may be that controller 105 is configured to, in response to expiry of the third period of time, cause UVC light source 109 to emit far-UVC light at a different (for example, reduced) intensity to the further given intensity (for example, cease to emit far-UVC light) into the target environment for a fourth period of time. In embodiments, controller 105 is configured to operate timer 106 to monitor one or both of the third and fourth periods of time. Thus, in embodiments, operating lighting device 100 in the unoccupied target environment operating mode comprises controlling UVC light source 109 to emit far-UVC light at a second average power. Embodiments in which UVC light source 109 is configured to emit far-UVC light into the target environment for a third period of time and, in response to expiry of the third period of time, cause UVC light source 109 to cease to emit far-UVC light into the target environment for a fourth period of time, can reduce the amount of time for which UVC light source 109 is active, thereby preserving and extending the operating life of UVC light source 109.

In embodiments, one or more of the first period of time, the second period of time, the third period of time and the fourth period of time are predetermined (for example, having been set during manufacture or factory calibration).

In embodiments, operating lighting device 100 in the unoccupied target environment operating mode comprises repeating a predetermined number of times the causing the lighting device to emit far-UVC light at the further given intensity for the third period of time and the causing the lighting device to emit far-UVC light at the different intensity to the further given intensity for the fourth period of time. Thus, in embodiments, lighting device 100 is configured to, whilst operating in the unoccupied target environment operating mode, perform a predetermined number of cycles of activating and deactivating UVC light source 109. As previously mentioned, by repeatedly activating and deactivating UVC light source 109 it is possible to control an average power of far-UVC light emission by UVC light source 109. Thus, in embodiments, operating lighting device 100 in the unoccupied target environment operating mode comprises controlling UVC light source 109 to emit far-UVC light at a second average power for a period of time. In embodiments, the second average power is greater than the first average power. In embodiments, the second average power exceeds a predetermined maximum dosage rate which is deemed safe.

In embodiments, controller 105 is configured to, in response to the monitoring indicating the presence of one or more people in the target environment whilst the lighting device is operating in the unoccupied target environment operating mode, cause lighting device 100 to revert to operate in the occupied target environment operating mode. Reverting to the occupied target environment operating mode in response to the monitoring indicating the presence of one or more people in the target environment prevents lighting device 100 emitting far-UVC light in such a way that people present in the target environment are exposed to an excess dosage of far-UVC light.

In embodiments, controller 105 is configured to, in response to the monitoring indicating that the target environment has been vacant for a fifth period of time, cause the lighting device to operate in an idle operating mode. In such embodiments, it may be that controller 105 is configured to operate timer 106 to monitor the fifth period of time. In alternative embodiments, controller 105 is configured to cause the lighting device to operate in the idle operating mode in response to completion of a predetermined number of cycles of activating and deactivating UVC light source 109. In embodiments, the fifth period of time is predetermined (for example, having been set during manufacture or factory calibration).

In embodiments, operating lighting device 100 in the idle operating mode comprises causing UVC light source 109 to cease to emit far-UVC light. In embodiments, operating lighting device 100 in the idle operating mode comprises causing no far-UVC light to be emitted into the target environment by the lighting device. Operating lighting device 100 in an idle operating mode reduces the amount of time UVC light source 109 is active, thereby preserving and extending the life of UVC light source 109.

In embodiments, the fifth period of time is longer than the first period of time. In embodiments, the fifth period of time is longer than the second period of time. In embodiments, the fifth period of time is longer than the third period of time. In embodiments, the fifth period of time is longer than the fourth period of time. In embodiments, the fifth period of time is longer than each of the first, second, third and fourth periods of time. In embodiments, the fifth period of time is longer than (for example, at least double) the sum of the third and fourth periods of time. In alternative embodiments, the fifth period of time is shorter than one or more (for example, all) of the first, second, third, and fourth periods of time.

Lighting device 100 comprises a processor 121 and an associated memory 123. It may be that some or all of the functionality of occupancy sensor 101, controller 105, UVC light source 109, transceiver 113, and distance sensor 117 is implemented partially or wholly by the processor 121 (for example, by executing instructions stored in the memory 123).

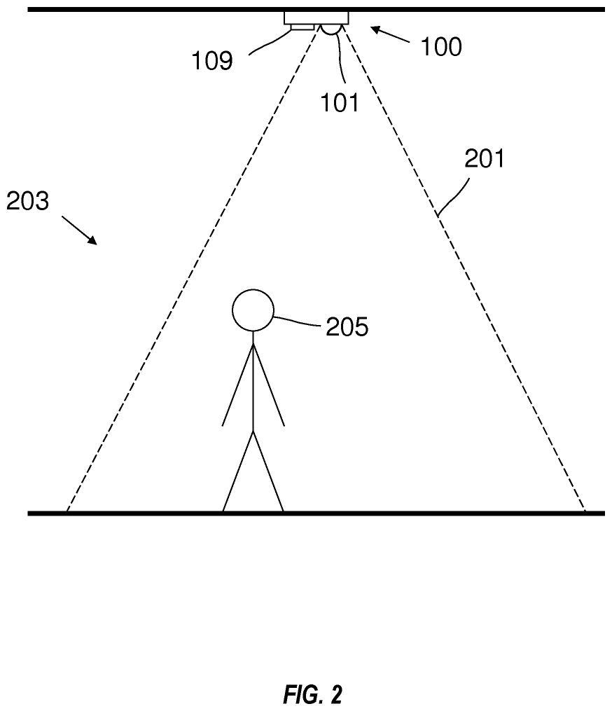

FIG. 2 shows a schematic view of an example installed lighting device 100 according to embodiments of the present disclosure. In these embodiments, lighting device 100 (including occupancy sensor 101 and UVC light source 109) comprises a single unit mounted to a ceiling. Occupancy sensor 101 has a field of view 201, which defines a target environment 203 within which occupancy sensor 101 is configured to monitor for the presence of one or more people 205.

In the embodiments illustrated by FIG. 2, occupancy sensor 101 forms part of lighting device 100. However, in alternative embodiments, occupancy sensor 101 is separate from the rest of lighting device 100 (for example, being in a separate unit or installation).

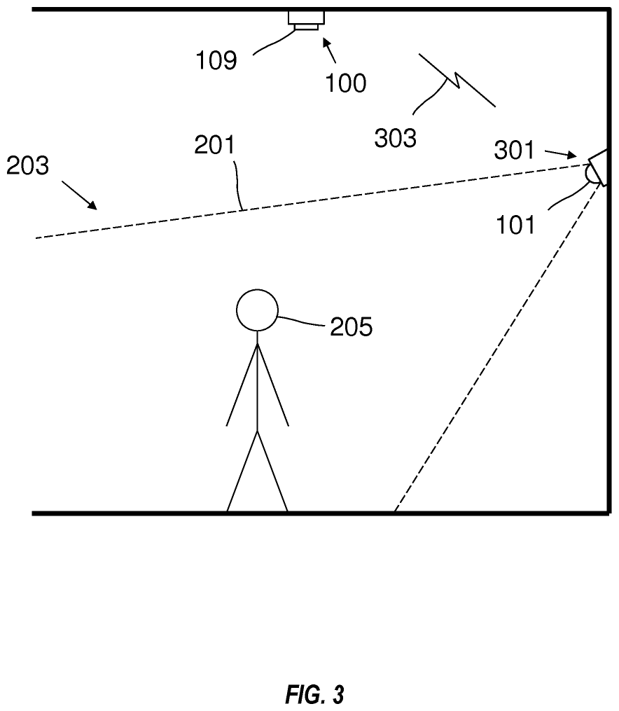

FIG. 3 shows a schematic view of a further example installed lighting device 100 according to embodiments of the present disclosure in which occupancy sensor 101 comprises a separate sensor unit 301 to that of lighting device 100. In such embodiments, it may be that occupancy sensor 101 is configured to communicate with lighting device 100 (for example, via a communications network) in order to communicate information on the occupancy of target environment 203 to lighting device 100. In embodiments, lighting device 100 and sensor unit 301 are configured to communicate with one another via a communication network. In embodiments, the communication network comprises a wired network. In embodiments the communication network comprises a wireless network 303. In other embodiments, multiple occupancy sensors are employed to monitor the target environment (for example, comprising multiple distinct and/or at least partially overlapping fields of view).

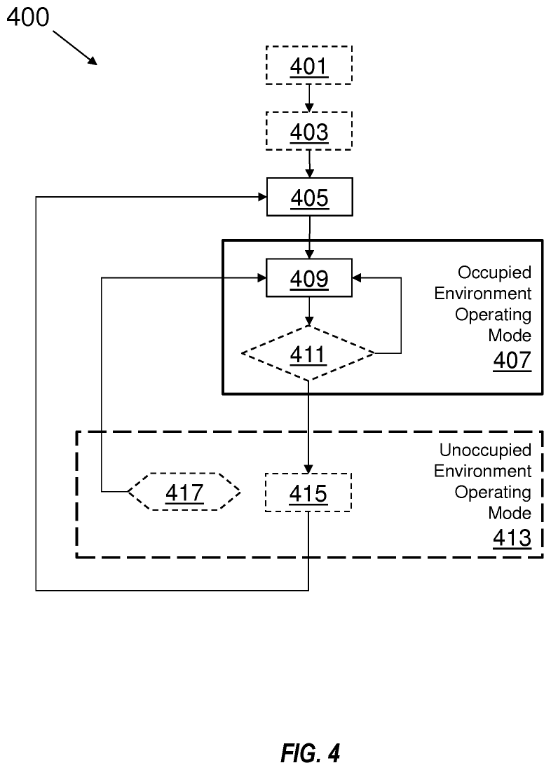

FIG. 4 shows a flow chart illustrating the steps of a method 400 of operating a germicidal lighting device according to embodiments of the present disclosure. In embodiments, the lighting device comprises an excimer lamp.

An optional first step of method 400, represented by item 401, comprises receiving an indication of one or more dimensions of the target environment. In embodiments, the receiving comprises operating one or more sensors to determine the one or more dimensions. In such embodiments, it may be that the one or more sensors comprise one or more of: a LiDAR sensor, a PIR sensor, an ultrasonic sensor, and a sonar sensor. In embodiments, the receiving comprise s receiving user input indicative of the one or more dimensions.

An optional second step of method 400, represented by item 403, comprises determining a first period of time and a second period of time on the basis of the indicated one or more dimensions.

A third step of method 400, represented by item 405, comprises monitoring a target environment for one or more people. In embodiments, the monitoring comprises operating an occupancy sensor. In such embodiments, it may be that the occupancy sensor comprises one or more of: a LiDAR sensor, a PIR sensor, an ultrasonic sensor, and a sonar sensor.

A fourth step of method 400, represented by item 407, comprises in response to the monitoring indicating the presence of one or more people, causing the lighting device to operate in an occupied target environment operating mode. Operating in the occupied target environment operating mode comprises a step, represented by item 409, of causing the lighting device to emit far-UVC light at a given intensity into the target environment for the first period of time, and, in response to expiry of the first period of time, causing the lighting device to emit far-UVC light at a different (for example, reduced) intensity to the given intensity (for example, by ceasing to emit far-UVC light) into the target environment for the second period of time.

In embodiments, at least one of the first period of time and the second period of time is dependent on a dimension of the target environment. In embodiments, at least one of the first period of time and the second period of time is dependent on an intensity of far-UVC light emission by the lighting device. In embodiments, at least one of the first period of time and the second period of time is dependent on a predetermined maximum far-UVC light dosage rate. In such embodiments, it may be that the predetermined maximum far-UVC light dosage rate is 3mj/cm²/hr. In embodiments, the first period of time and the second period of time are each longer than 0.1 seconds. In embodiments, the ratio of the first period of time to the second period of time is no greater than 1:1. In embodiments, emitting far-UVC light comprises emitting light having a wavelength of 222 nm. In embodiments, emitting far-UVC light comprises emitting light predominantly (for example, substantially only) having a wavelength of 222 nm.

In embodiments, operating in the occupied target environment operating mode further comprises an optional step, represented by item 411, of, in response to the expiry of the second period of time, repeating the monitoring the target environment for one or more people. In such embodiments, it may be that method 400 comprises, in response to the monitoring indicating the continued presence of one or more people, repeating item 409. Thus, in embodiments, method 400 comprises, in response to the monitoring continuing to indicate the presence of one or more people, causing the lighting device to continue to operate in the occupied target environment operating mode by repeating the causing the lighting device to emit far-UVC light at the given intensity for the first period of time and the causing the lighting device to emit far-UVC light at the different intensity to the given intensity (for example, by ceasing to emit far-UVC light) for the second period of time.

An optional fifth step of method 400, represented by item 413 comprises, in response to the monitoring indicating that the one or more people have vacated the target environment, causing the lighting device to operate in an unoccupied target environment operating mode. In embodiments, an intensity of far-UVC light emission by the lighting device in the occupied target environment operating mode is lower than an intensity of far-UVC light emission by the lighting device in the unoccupied target environment operating mode. In embodiments, operating in the unoccupied target environment mode comprises causing the lighting device to emit far-UVC light at a maximum intensity.

In embodiments, far-UVC light emission by the lighting device in the unoccupied target environment operating mode differs from far-UVC light emission by the lighting device in the occupied target environment operating mode. In embodiments, operating in the unoccupied target environment operating mode comprises an optional step, represented by item 415, of causing the lighting device to emit far-UVC light at a further given intensity into the target environment for a third period of time and, in response to expiry of the third period of time, causing the lighting device to emit far-UVC light at a different (for example, reduced) intensity to the further given intensity (for example, by ceasing to emit far-UVC light) into the target environment for a fourth period of time. In embodiments, operating the lighting device in the unoccupied target environment operating mode comprises repeating a predetermined number of times the causing the lighting device to emit far-UVC light at the further given intensity for the third period of time and the causing the lighting device to emit far-UVC light at the different intensity to the further given intensity (for example, by ceasing to emit far-UVC light) for the fourth period of time.

In embodiments, method 400 comprises an optional step, represented by item 417, of, in response to the monitoring indicating the presence of one or more people whilst the lighting device is operating in the unoccupied target environment operating mode, interrupting the unoccupied target environment operating mode. In embodiments, interrupting the unoccupied target environment operating mode comprises causing the lighting device to revert to operate in the occupied target environment operating mode.

In embodiments, method 400 comprises an optional step (not shown) of, in response to the monitoring indicating that the target environment has been vacant for a fifth period of time, causing the lighting device to operate in an idle operating mode. In embodiments, operating the lighting device in the idle operating mode comprises operating the lighting device such that no far-UVC light is emitted into the target environment by the lighting device. In embodiments, the fifth period of time is longer than each of the first, second, third and fourth periods of time.

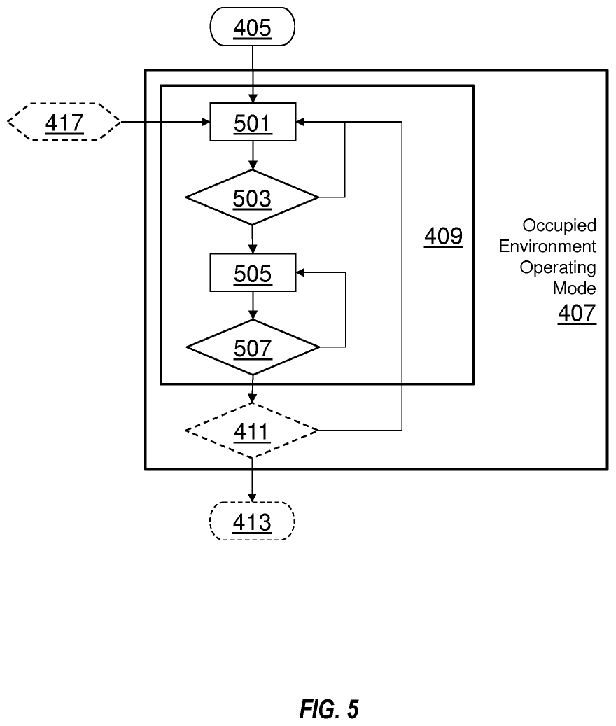

FIG. 5 shows a flow chart illustrating the occupied target environment operating mode of FIG. 4 according to embodiments of the present disclosure.

In embodiments, the lighting device begins operating in occupied target environment operating mode 407 in response to the monitoring indicating the presence of one or more people (for example, either whilst the lighting device is operating in idle operating mode 405 or in unoccupied target environment operating mode 413 in response to triggering of interrupt 417).

In embodiments, a first step, represented by item 501, of operation in the occupied target environment operating mode comprises causing the lighting device to emit far-UVC light at a given intensity into the target environment for a first period of time. Thus, in embodiments, a second step, represented by item 503, comprises waiting for the first period of time.

In embodiments, a third step, represented by item 505 and triggered in response to expiry of the first period of time, comprises causing the lighting device to emit far-UVC light at a different (for example, reduced) intensity to the given intensity (for example, by ceasing to emit far-UVC light) into the target environment for a second period of time. Thus, in embodiments, a fourth step, represented by item 507, comprises waiting for the second period of time.

In embodiments, a fifth step, represented by item 411 and triggered in response to expiry of the first period of time, comprises monitoring the target environment for one or more people. In embodiments, method 400 comprises, in response to monitoring 411 indicating the presence of one or more people in the target environment, continuing to operate in occupied target environment operating mode 407 by returning to the first step 501. In embodiments, method 400 comprises, in response to monitoring 411 indicating that no people are present in the target environment, causing the lighting device to operate in unoccupied target environment operating mode 413.

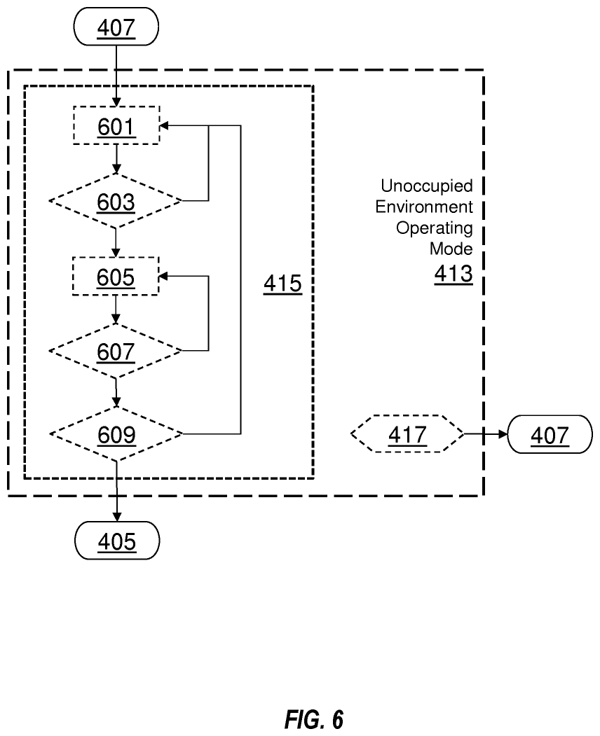

FIG. 6 shows a flow chart illustrating the unoccupied target environment operating mode of FIG. 4 according to embodiments of the present disclosure.

In embodiments, the lighting device begins operating in unoccupied target environment operating mode 413 in response to the monitoring indicating that the one or more people have vacated the target environment. In embodiments, the lighting device begins operating in unoccupied target environment operating mode 413 only after (for example, immediately after) having operated in occupied target environment operating mode 407.

In embodiments, a first step, represented by item 601, of operation in the unoccupied target environment operating mode comprises causing the lighting device to emit far-UVC light at a further given intensity into the target environment for a third period of time. Thus, in embodiments, a second step, represented by item 603, comprises waiting for the third period of time.

In embodiments, a third step, represented by item 605 and triggered in response to expiry of the third period of time, comprises causing the lighting device to emit far-UVC light at a different (for example, reduced) intensity to the further given intensity (for example, by ceasing to emit far-UVC light) into the target environment for a fourth period of time. Thus, in embodiments, a fourth step, represented by item 607, comprises waiting for the fourth period of time.

In embodiments, operating the lighting device in the unoccupied target environment operating mode comprises repeating steps 601, 603, 605, and 607 for a predetermined number of cycles. Thus, in embodiments, a fifth step, represented by item 609, comprises determining whether the predetermined number of cycles have been completed. In embodiments, operating the lighting device in the unoccupied target environment operating mode comprises, in response to the predetermined number of cycles not have been completed, returning to step 601. In embodiments, operating the lighting device in the unoccupied target environment operating mode comprises, in response to the predetermined number of cycles having been completed, causing the lighting device to operate in idle operating mode 405. In alternative embodiments, operating the lighting device in the unoccupied target environment operating mode comprises, in response to the monitoring indicating that the target environment has been vacant for a fifth period of time, causing the lighting device to operate in idle operating mode 405.

In embodiments, method 400 comprises, in response to the monitoring indicating the presence of one or more people whilst the lighting device is operating in unoccupied target environment operating mode 413, interrupting the unoccupied target environment operating mode, represented by item 417, (for example, by causing the lighting device to revert to operate in occupied target environment operating mode 407). In embodiments, the triggering of interrupt 417 causes lighting device to immediately cease to operate in unoccupied target environment operating mode 413.

Whilst the present disclosure has been described and illustrated with reference to particular embodiments, it will be appreciated by those of ordinary skill in the art that the present disclosure lends itself to many different variations not specifically illustrated herein. By way of example only, certain possible variations will now be described.

Whilst in the illustrated embodiments lighting device 100 comprises only a single occupancy sensor 101, it will be appreciated that, in alternative embodiments, lighting device 100 may comprise multiple occupancy sensors. In such embodiments, the multiple occupancy sensor may be of the same type or of different types. In embodiments, each of the multiple occupancy sensors comprises a different field of view. Thus, in embodiments, the multiple occupancy sensors are configured to monitor different parts of the target environment. In embodiments, a field of view of a first sensor of the multiple occupancy sensors is configured to at least partially overlap that of a second occupancy sensor.

Whilst in the illustrated embodiments lighting device 100 comprises only a single UVC light source 109, it will be appreciated that, in alternative embodiments, lighting device 100 may comprise multiple UVC light sources. In embodiments, each of the multiple UVC light sources is configured to emit far-UVC light into a different area. In embodiments, the multiple UVC light sources are configured to each emit far-UVC light into different parts of the target environment.

In embodiments, occupancy sensor 101 is part of lighting device 100. However, in alternative embodiments, occupancy sensor 101 may be separate from lighting device 100 (for example, part of a separate unit). In such embodiments, it may be that occupancy sensor 101 is located apart from lighting device 100. In some situations, locating occupancy sensor 101 apart from lighting device 100 may allow improved monitoring of the target environment.

Whilst the illustrated embodiments show lighting devices 100 mounted to a ceiling and a wall, it will be appreciated by the skilled person that other mounting arrangements are also possible. For example, in alternative embodiments, the lighting device (or one or more parts thereof) is suspended from a ceiling. In embodiments, the lighting device (or one or more parts thereof) is mounted on a wall, or a stand or other lighting fixture.

It will be appreciated that the lighting device 100 may comprise one or more processors and/or memory. Thus, in embodiments, lighting device 100 comprises a processor 121 and an associated memory 123. Processor 121 and associated memory 123 may be configured to perform one or more of the above-described functions of lighting device 100. Each device, module, component, machine or function as described in relation to any of the examples described herein (for example, occupancy sensor 101, controller 105, UVC light source 109, transceiver 113, and distance sensor 117) may similarly comprise a processor or may be comprised in apparatus comprising a processor. One or more aspects of the embodiments described herein comprise processes performed by apparatus. In some examples, the apparatus comprises one or more processors configured to carry out these processes. In this regard, embodiments may be implemented at least in part by computer software stored in (non-transitory) memory and executable by the processor, or by hardware, or by a combination of tangibly stored software and hardware (and tangibly stored firmware). Embodiments also include computer programs, particularly computer programs on or in a carrier, adapted for putting the above-described embodiments into practice. The program may be in the form of non-transitory source code, object code, or in any other non-transitory form suitable for use in the implementation of processes according to embodiments. The carrier may be any entity or device capable of carrying the program, such as a RAM, a ROM, or an optical memory device, etc.

The one or more processors of the lighting device 100 may comprise a central processing unit (CPU). The one or more processors may comprise a graphics processing unit (GPU). The one or more processors may comprise one or more of a field programmable gate array (FPGA), a programmable logic device (PLD), or a complex programmable logic device (CPLD). The one or more processors may comprise an application specific integrated circuit (ASIC). It will be appreciated by the skilled person that many other types of device, in addition to the examples provided, may be used to provide the one or more processors. The one or more processors may comprise multiple co-located processors or multiple disparately located processors. Operations performed by the one or more processors may be carried out by one or more of hardware, firmware, and software.

The one or more processors may comprise data storage. The data storage may comprise one or both of volatile and non-volatile memory. The data storage may comprise one or more of random access memory (RAM), read-only memory (ROM), a magnetic or optical disk and disk drive, or a solid-state drive (SSD). It will be appreciated by the skilled person that many other types of memory, in addition to the examples provided, may also be used. It will be appreciated by a person skilled in the art that the one or more processors may each comprise more, fewer and/or different components from those described.

The techniques described herein may be implemented in software or hardware, or may be implemented using a combination of software and hardware. They may include configuring an apparatus to carry out and/or support any or all of techniques described herein. Although at least some aspects of the examples described herein with reference to the drawings comprise computer processes performed in processing systems or processors, examples described herein also extend to computer programs, for example computer programs on or in a carrier, adapted for putting the examples into practice. The carrier may be any entity or device capable of carrying the program. The carrier may comprise a computer readable storage media. Examples of tangible computer-readable storage media include, but are not limited to, an optical medium (e.g., CD-ROM, DVD-ROM or Blu-ray), flash memory card, floppy or hard disk or any other medium capable of storing computer-readable instructions such as firmware or microcode in at least one ROM or RAM or Programmable ROM (PROM) chips.

Where in the foregoing description, integers or elements are mentioned which have known, obvious or foreseeable equivalents, then such equivalents are herein incorporated as if individually set forth. Reference should be made to the claims for determining the true scope of the present disclosure, which should be construed so as to encompass any such equivalents. It will also be appreciated by the reader that integers or features of the present disclosure that are described as preferable, advantageous, convenient or the like are optional and do not limit the scope of the independent claims. Moreover, it is to be understood that such optional integers or features, whilst of possible benefit in some embodiments of the present disclosure, may not be desirable, and may therefore be absent, in other embodiments.

What is claimed is:

1. A method of operating a germicidal lighting device, the method comprising:
monitoring a target environment for one or more people; and
in response to the monitoring indicating the presence of one or more people, causing the germicidal lighting device to operate in an occupied target environment operating mode comprising:
causing the germicidal lighting device to emit far-UVC light at a given intensity into the target environment for a first period of time; and
in response to expiry of the first period of time, causing the germicidal lighting device to emit far-UVC light at a different intensity to the given intensity into the target environment for a second period of time.

2. The method of claim 1, further comprising, in response to the monitoring continuing to indicate the presence of one or more people, causing the germicidal lighting device to continue to operate in the occupied target environment operating mode by repeating the causing the germicidal lighting device to emit far-UVC light at the given intensity for the first period of time and the causing the germicidal lighting device to emit far-UVC light at the different intensity to the given intensity for the second period of time.

3. The method of claim 1, wherein:
at least one of the first period of time and the second period of time is dependent on a dimension of the target environment, and/or
at least one of the first period of time and the second period of time is dependent on an intensity of far-UVC light emission by the germicidal lighting device, and/or
at least one of the first period of time and the second period of time is dependent on a predetermined maximum far-UVC light dosage rate.

4. The method of claim 3, wherein the predetermined maximum far-UVC light dosage rate is as determined by a safety regulation.

5. The method of claim 1, further comprising:
receiving an indication of one or more dimensions of the target environment; and
determining the first period of time and the second period of time on the basis of the indicated one or more dimensions.

6. The method of claim 5, wherein:
the method further comprises operating one or more sensors to determine the one or more dimensions, or
the receiving comprises receiving user input indicative of the one or more dimensions.

7. The method of claim 1, comprising, in response to the monitoring indicating that the one or more people have vacated the target environment, causing the germicidal lighting device to operate in an unoccupied target environment operating mode wherein far-UVC light emission by the germicidal lighting device differs from far-UVC light emission in the occupied target environment operating mode.

8. The method of claim 7, wherein an intensity of far-UVC light emission by the germicidal lighting device in the occupied target environment operating mode is lower than an intensity of far-UVC light emission by the germicidal lighting device in the unoccupied target environment operating mode.

9. The method of claim 7, wherein operating in the unoccupied target environment mode comprises causing the germicidal lighting device to emit far-UVC light at a maximum intensity.

10. The method of claim 7, wherein operating the germicidal lighting device in the unoccupied target environment operating mode comprises:
causing the germicidal lighting device to emit far-UVC light at a further given intensity into the target environment for a third period of time; and
in response to expiry of the third period of time, causing the germicidal lighting device to emit far-UVC light at a different intensity to the further given intensity into the target environment for a fourth period of time.

11. The method of claim 10, wherein operating the germicidal lighting device in the unoccupied target environment operating mode comprises repeating a predetermined number of times the causing the germicidal lighting device to emit far-UVC light at the further given intensity for the third period of time and the causing the germicidal lighting device to emit far-UVC light at the different intensity to the further given intensity for the fourth period of time.

12. The method of claim 7, comprising, in response to the monitoring indicating the presence of one or more people whilst the germicidal lighting device is operating in the unoccupied target environment operating mode, causing the germicidal lighting device to revert to operate in the occupied target environment operating mode.

13. The method of claim 1, comprising, in response to the monitoring indicating that the target environment has been vacant for a fifth period of time, causing the germicidal lighting device to operate in an idle operating mode wherein no far-UVC light is emitted into the target environment by the germicidal lighting device.

14. The method of claim 13, wherein the fifth period of time is longer than each of the first, second, third and fourth periods of time.

15. The method of claim 1, wherein:
the first period of time and the second period of time are each longer than 0.1 seconds, and/or
a ratio of the first period of time to the second period of time is no greater than 1:1.

16. The method of claim 1, comprising communicating, by the germicidal lighting device, with one or more further germicidal lighting devices or other devices for uses including processing and storage of data and coordination of the devices.

17. The method of claim 1, wherein causing the germicidal lighting device to emit far-UVC light at the different intensity comprises causing the germicidal lighting device to cease to emit light.

18. A germicidal lighting device comprising:
a UVC light source configured to emit far-UVC light;
an occupancy sensor configured to monitor a target environment for one or more people; and
a controller configured to, in response to the monitoring indicating the presence of one or more people, cause the germicidal lighting device to operate in an occupied target environment operating mode comprising:
causing the germicidal lighting device to emit far-UVC light at a given intensity into the target environment for a first period of time; and
in response to expiry of the first period of time, causing the germicidal lighting device to emit far-UVC light at a different intensity to the given intensity into the target environment for a second period of time.

19. The device of claim 18, wherein the light source comprises an excimer lamp.

20. A non-transitory computer-readable storage medium comprising computer-executable instructions which, when executed by a processor, cause a computerized device to perform a method of operating a germicidal lighting device, the method comprising:
monitoring a target environment for one or more people; and
in response to the monitoring indicating the presence of one or more people, causing the germicidal lighting device to operate in an occupied target environment operating mode comprising:
causing the germicidal lighting device to emit far-UVC light at a given intensity into the target environment for a first period of time; and
in response to expiry of the first period of time, causing the germicidal lighting device to emit far-UVC light at a different intensity to the given intensity into the target environment for a second period of time.
